(19) United States
(12) Patent Application Publication
(10) Pub. No.: US 2025/0325220 A1
(43) Pub. Date: Oct. 23, 2025

(54) DYNAMIC BALANCE ASSESSMENT AND TRAINING DEVICE

(71) Applicant: Mark Thomas Rowley, NORWALK, CT (US)

(72) Inventor: Mark Thomas Rowley, NORWALK, CT (US)

(21) Appl. No.: 18/639,889

(22) Filed: Apr. 18, 2024

(51) Int. Cl. A61B 5/00; A61B 5/103; A61B 5/11; A63B 26/00

(52) U.S. Cl. CPC A61B 5/4023; A61B 5/1036; A61B 5/1126; A61B 5/702; A63B 26/003

(57) ABSTRACT

A dynamic balance measurement and training device provides an efficient, portable, and versatile solution for dynamic balance measurement and training. Three detachable rods are arranged in a ‘Y’ shape around a main platform. A movable sliding box, guided by the patient's leg movement, is configured to move along the detachable rods. The device includes laser distance sensors for accurate, automated balance measurement, as well as a microcontroller configured to process the laser distance sensor measurements and calculate balance scores, target distances, and other relevant information. These data can be recorded and may be displayed on a built-in screen or transmitted wirelessly for further analysis and tracking. An integrated pressure sensor provides feedback to the patient or clinician, indicating when assessments should be redone due to missteps or loss of balance. The device offers an integrated solution for balance assessment and training, promoting patient engagement and improving clinical efficiency.

### BACKGROUND OF THE INVENTION

### Field of the Invention

The present invention relates generally to devices and apparatus for assessing dynamic balance and mobility. In particular, the present invention relates to a portable, automated three direction dynamic balance assessment device.

### Description of the Related Art

Dynamic balance is generally measured by the Star Excursion Balance Test (“SEBT”) using commercially available devices like the Y Balance Test Kit® (Functional Movement Systems, Inc.). The Y Balance Test Kit provides three rods to permit the patient to use one foot to push a slider along in forward medial and rearward lateral directions, while using their other foot to stand on a central main platform. These tests assess and quantify balance by measuring the distance an individual can reach by moving one foot in multiple directions while maintaining their balance on their other foot at a fixed point, and are recognized as being reliable and valid tools for measuring dynamic balance.

While currently known dynamic balance measurement devices are capable of a wide variety of applications, there is a continuing demand for devices that are more portable, more versatile and more efficient. In particular, it is important that these devices work on different types of flooring without added friction, to ensure that assessments have greater accuracy and are more reproducible. In addition, it is desirable to provide automatic measurement capabilities that improve both efficiency and accuracy.

Furthermore, the inventor has determined interference between the sliders and the flooring material upon which the dynamic balance measuring device is placed can result in assessments that are inconsistent from facility to facility. Accordingly, it would be further beneficial to provide a dynamic balance measuring device where the type of flooring in the facility does not affect the effort required to move the sliders.

Conventional dynamic balance and training typically requires using different devices or systems for balance measurement and training, leading to a more disjointed and less efficient process. Accordingly, it would be desirable to integrate disparate balance training features into a single dynamic balance assessment device, allowing both patients and clinicians to more easily work towards achieving specific distance targets.

Additionally, the incorporation of so-called “smart” technologies, such as real-time balance scores, target distances, and other relevant information display, as well as wired or wireless data tracking and integration with external devices or applications, would greatly benefit both clinicians and patients. These features are currently lacking in conventional dynamic balance measurement devices.

Furthermore, it would be beneficial to provide a mechanism that alerts the patient or clinician when a test should be repeated (e.g. due to a misstep or a loss of balance, etc.) as well as a mechanism for guiding the test administration process, so as to enhance the overall utility and effectiveness of the dynamic balance assessment device.

### SUMMARY OF THE INVENTION

It is one object of the present invention to provide a dynamic balance measurement device that is highly compatible with various types of flooring.

It is another object of the present invention to provide a dynamic balance measurement device with a positively retained, detachable rod mechanism so as to enhance portability, while ensuring versatility and ease of use.

It is another object of the present invention to provide a dynamic balance measurement device that accurately and automatically measures the patient's balance and range.

It is still another object of the present invention to provide a dynamic balance measuring device with a training feature that allows the patient to more easily achieve reaching a specific distance goal.

These objects and others are achieved by an embodiment of the present invention, which provides a dynamic balance measurement device including a main platform with detachable rods defining a “Y” shape. The rods each guide a sliding box that is displaced by the patient's leg movement along the length of the rods. The detachable rods are configured to support the sliding boxes so they can desirably be displaced without contacting the ground, thus minimizing or eliminating friction with flooring such as carpet and the like. Additionally, the rods and sliding box may beneficially provide bearing surfaces to effectively preclude resistance when the sliding box is displaced along the rod.

In accordance with another embodiment of the present invention, the dynamic balance measurement device includes laser distance sensors with a microcontroller configured to process measurements obtained from the laser distance sensors. By these features, the dynamic balance measurement device can calculate data such as balance scores, target distances, and other relevant information which may be displayed on a built-in screen, transmitted by wire to a separate display, or transmitted wirelessly via Bluetooth. These data can be saved and made available for further analysis and tracking.

Still another embodiment of the present invention provides a dynamic balance measurement device that includes a pressure sensor integrated into the main platform, and/or sliding boxes. The pressure sensor(s) are monitored by the microcontroller and, using the built-in screen or other wired or wireless display, provide feedback to inform when the test or evaluation should be redone, for instance due to patient missteps or loss of balance. The dynamic balance measurement device may also use a built-in screen, or wired or wireless external display to guide the test administration process, as well as to alert the operator if a measurement significantly deviates from the norm so as to merit further analyses.

### BRIEF DESCRIPTION OF DRAWING

FIG. 1 is a perspective view of the side of a first embodiment of the present invention;

FIG. 2 is an elevation view of the embodiment shown in FIG. 1;

FIGS. 3 and 4 are partial cutaway elevation views of the embodiment shown in FIG. 1;

FIG. 5 is a partial elevation view of an embodiment of the rod and moveable slider of the present invention;

FIG. 6 is a perspective view of another embodiment of the rod and moveable slider of the present invention;

FIGS. 7 and 8 are respectively side and elevation views of an additional embodiment of the rod and moveable slider of the present invention; and

FIGS. 9 and 10 are additional alternative embodiments of the rod and moveable slider of the present invention.

### DESCRIPTION OF THE EMBODIMENTS

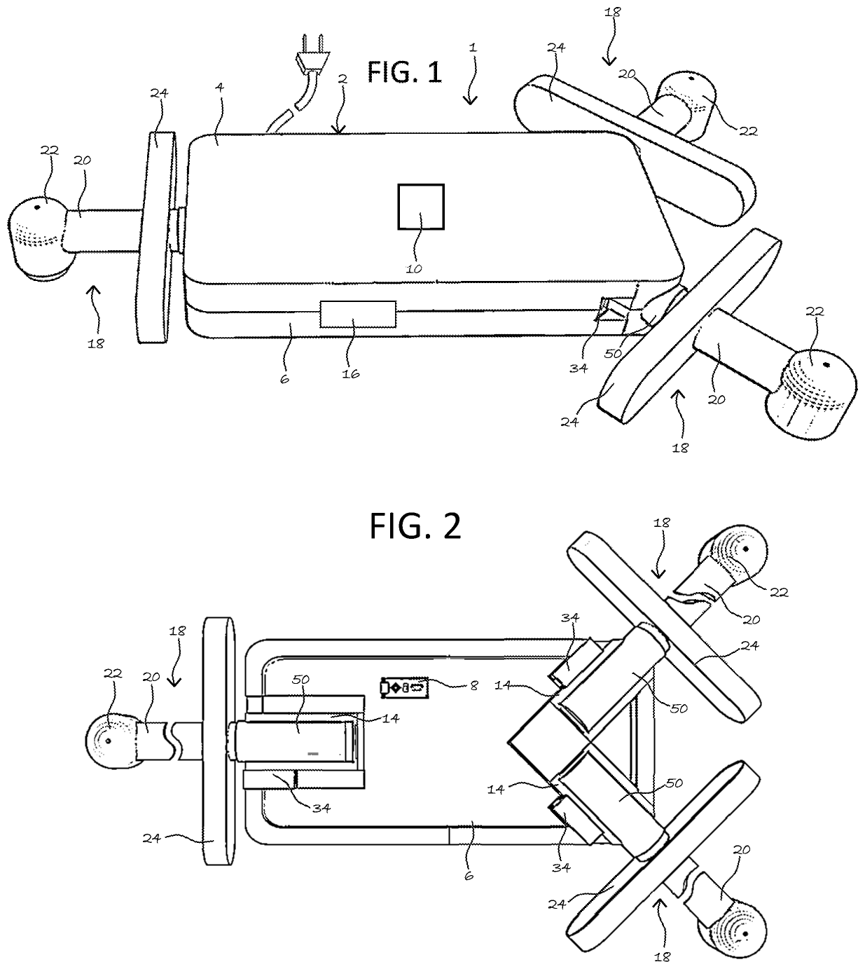

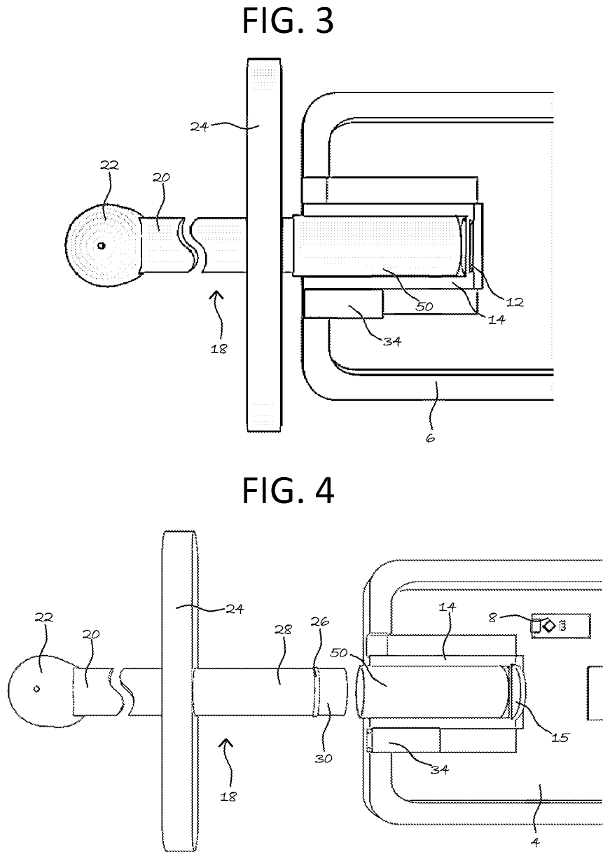

Turning to the drawings, where like numerals denote like components across the different views, the balance assessment and training device 1, shown in FIG. 1 provides an enclosure 2 designed to bear a patient's weight. Enclosure 2 is formed by enclosure top 4 oriented above enclosure bottom 6. As illustrated in FIG. 2, enclosure bottom 6 includes internal mounting points for various components, including microcontroller board 8, proximal magnet slots 12 (FIG. 3), and rod support housings 14. Enclosure top 4 provides pressure sensor 10 and LCD Screen 16.

Balance assessment and training device 1 further includes rod and slider assemblies 18, also shown in the Figures. In a first embodiment best illustrated in FIGS. 3 and 4, each rod and slider assembly 18 is composed of a rod 20, a distal rod end cap 22, moveable slider 24, and an internal magnet 26. Internal magnet 26 is retained at the proximal end 28 of rod 20 by proximal rod end cap 30. Balance assessment and training device 1 is assembled by inserting proximal end 28 of rod 20 into sleeve 50 within rod support housing 14, where internal magnet 26 magnetically interacts with platform magnet 15 retained within magnet slot 12 to removably retain rod and slider assembly 18.

This configuration permits the free-moving slider 24 to be manipulated by a user's foot, with rod and slider assembly 18 being retained within enclosure 2 and secured against unintentional disconnection by platform magnet 15 and internal magnet 26, yet still enabling removal of rod and slider assembly 18 without tools by outwardly dislocating rod 20.

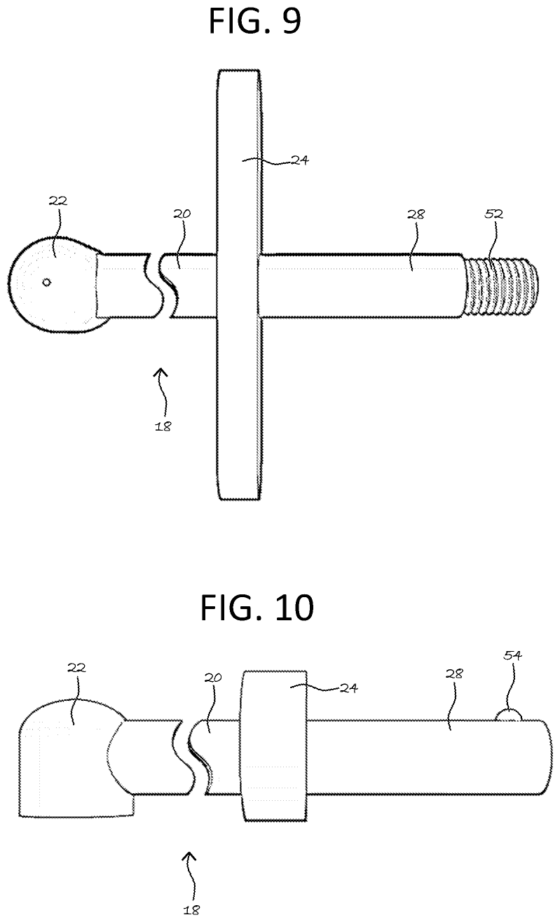

In other suitable embodiments, rod 20 may be retained within enclosure 2 by providing an interference fit of proximal rod end 28 and sleeve 50. Similarly, proximal rod end 28 and sleeve 50 may provide complimentary grooved threads 52 (FIG. 9), which are configured to permit detachably retaining rod 20 within said central platform by threading the proximal rod end 28 into sleeve 50 thereby retaining rod 20 within rod support housing 14. Likewise, proximal rod end 28 and sleeve 50 may utilize a non-interference (e.g., looser) fit in which proximal rod end 28 provides an outwardly and axially biased protruding button 54 (FIG. 10) configured to fit into a corresponding aperture (unillustrated) located within sleeve 50 and rod support housing 14.

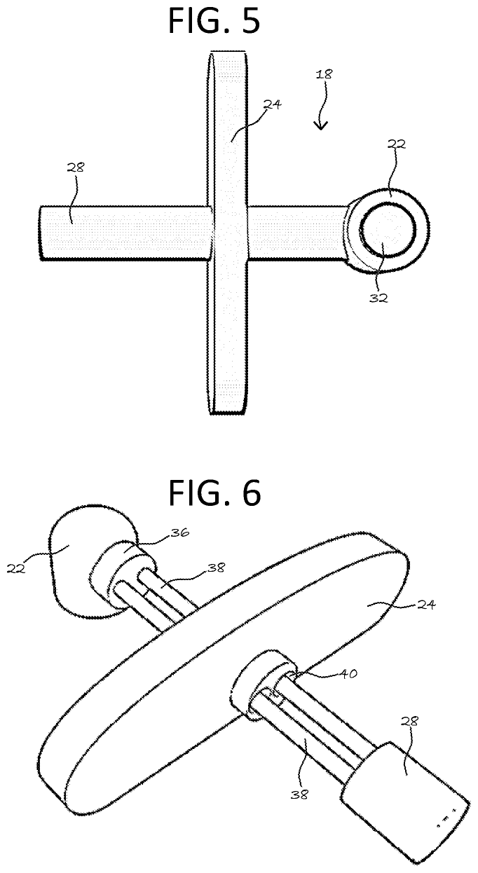

As illustrated in FIG. 1, a protruding portion 23 of distal rod end cap 22 extends axially beyond rod 20 so as to elevate rod and slider assembly 18 off the ground, thereby facilitating the motion of moveable slider 24 along rod 20 without friction or interference from flooring, carpets, rugs and the like. As depicted, protruding portion 23 may be configured to extend axially from rod 20 in opposing directions whereby protruding portion 23 extends axially further from rod 20 in one direction than the other direction. This arrangement effectively permits orienting protruding portion 23 so as to elevate moveable slider 24 in different heights from the flooring, e.g., depending on which of the two sides of protruding portion 23 contacts the floor. Accordingly, the present invention permits raising the rod 20 the minimum height necessary to preclude interference of moveable slider 24 from carpeting or rugs. Additionally, the outer end of protruding portion 23 is preferably equipped with a non-skid rubber or elastomeric foot 32 (FIG. 5) to preclude unintentional disengagement of internal magnet 26 and platform magnet 15 and thereby prevent inadvertently displacing rod 20 from rod support housing 14 during use.

As may be depicted in the Figures, rods 20 are illustrated as truncated for the purpose of showcasing the entire rod and slider assemblies 18. However, as generally used, rods 20 may desirably be approximately 3-4 feet long.

### Desirable Operation

To initially set up balance assessment and training device 1, internal magnet 26 is inserted within rod 20 and retained therein by installing proximal rod end cap 30. Platform magnet 15 is likewise inserted into magnet slot 12, and enclosure top 4 is then affixed to enclosure bottom 6. The proximal rod end 28 of each rod 20 is then inserted into its respective rod support housing 14, and the power cord is plugged into a suitable wall outlet. If desired, the power cord may be replaced with an internal or external battery supply (unillustrated). If provided, a power switch (unillustrated) is turned “on”, thereby energizing balance assessment and training device 1.

Balance assessment and training device 1 provides a laser distance sensor 34 for each moveable slider 24. Laser distance sensors 34 are configured to determine the distance moveable slider 24 has been displaced from enclosure 2 during a patient's assessment evaluation using emitted laser light. Each laser distance sensor 34 transmits the distance measurements of its corresponding moveable slider 24 to the microcontroller board 8.

Dynamic balance measurement device 1 desirably includes a pressure sensor 10 that is mounted on or underneath enclosure top 4. When a patient places their foot on enclosure 2, pressure sensor 10 is configured to detect the load from the patient's foot and relay this information to the microcontroller board 8.

The microcontroller board 8 is essentially the “brain” of the device and performs numerous functions and calculations that contribute to the overall functionality of the balance assessment and training device 1. The microcontroller board 8 processes the distance measurements for the movement of each of moveable sliders 24 along its respective rod 20, and can display these measurements in whole or in part on LCD screen 16, and may also be used to display other relevant information such as summaries or averages of these measurements. Similarly, the information display may include calculations, such as averaging multiple trials, or computing the percentage symmetry of movement attained by one leg is different directions, as well as the percentage symmetry of movement between the patient's two legs.

Microcontroller board 8 also monitors the reported load of the patient's foot from pressure sensor 10. A desirable function of the microcontroller board 8 is to use the data from pressure sensor 10 to identify and alert the operator or clinician about a patient's potential balance loss during a trial or evaluation. Potential balance loss can be determined when there is a sudden drop in the load measured by the pressure sensor 10 while the moveable sliders 24 are being manipulated, and so can reliably suggest that it may be appropriate for the operator or clinician to reevaluate or assess the patient.

For data tracking and logging purposes, the microcontroller board 8 can export the data to an external device, such as a computer or smart telephone. This feature allows for real-time monitoring of performance and progress.

The microcontroller board 8 may be equipped with advanced features that enhance the user experience and contribute to the efficacy of balance training. One such feature may include displaying distance targets on the LCD screen 16 or external device. This encourages users to displace moveable sliders 24 to specific positions along the rods 20, further promoting balance training. Moreover, the microcontroller board 8 can display instructions for the correct administration of tests on the LCD screen 16 or external device, thereby better providing users with a seamless and informative experience.

In use, the patient is positioned adjacent enclosure 2, aligning themselves such that one rod 20 is oriented directly in front, with the other two rods 20 situated behind, each of the rearward rods 20 being offset approximately 60 degrees from the frontward-oriented rod 20. The patient's weight is then supported on enclosure 2 using one foot. Pressure sensor 10 and laser distance sensors 34 are calibrated, zeroing out the system to account for both the initial position of moveable sliders 24 on rods 20. The patient is typically guided to execute an initial practice trial, placing their other foot on the side of moveable slider 24 and exhibiting force so as to push moveable slider 24 along its corresponding rod 20 without the patient losing balance, and while bearing minimal weight on the foot used to manipulate moveable slider 24.

After successfully completing the initial practice trial, the patient's dynamic balance may be assessed. For instance, the default evaluation mode preferably requires the patient to again balance on one foot, and to repeat pushing the frontward moveable slider 24 for a total of three separate times without loss of balance. These three successful trials are averaged, and the resulting mean is displayed on the LCD screen 16 or external device.

Pressure sensor 10 monitors force on enclosure 2 during the assessment, while laser distance sensor 34 monitors the displacement of moveable slider 24 along rod 20. If the patient stumbles, or loses his or her balance, as determined by a significant or abrupt reduction in load detected by the pressure sensor 10, microcontroller 8 causes display of a suitable instruction for the operator to repeat the trial on LCD screen 16. Additionally, microcontroller 8 can also be configured to monitor the quality of movement of moveable slider 24 to ensure its displacement along rod 20 by the patient is smooth, e.g., under control and with deliberate perceived intent. If the displacement of moveable slider 24 along rod 20 is not sufficiently smooth, LCD screen 16 may be similarly caused to display instruction for the operator to repeat the trial even in the absence of pressure sensor 10 detecting any significant or abrupt reduction in load.

After the patient satisfactorily completes his or her assessment using the moveable slider 24 on frontward medial rod 20, the assessment will be continued by repeating the evaluation using the remaining moveable sliders 24 on their respective rods 20. Accordingly, the patient then repeats these operations for the remaining rearward left and right lateral moveable sliders 24, thus completing nine total successful evaluation trials. The averages of three successful trials for each rearward left and right lateral moveable slider 24 is again computed and displayed on the LCD screen 16.

If clinically indicated for assessment or training, these procedures are thereafter subsequently replicated with the patient standing on his or her other foot, resulting in a total of nine additional successful trials. Upon completion of these nine trials for each leg, the microcontroller board 8 may calculate and cause LCD screen 16 to display a percent or absolute difference for each of the three directions for each leg, presenting an objective measure of the patient's balance in different directions. Similarly, microcontroller board 8 may be used to display a percent or absolute difference in mobility between the patient's two legs.

As the patient engages in these trials, laser distance sensors 34 may be employed to continuously track the displacement of moveable sliders 24 along their respective rods 20. This real-time data, along with a verification signifying balance was successfully maintained by the patient, are displayed on the built-in LCD screen 16. The balance assessment and training device 1 may offer various modes for testing or training, selectable via the LCD screen 16 interface.

Alternative modes may instruct the patient to execute only one trial in each direction, or provide the patient with specific target distances to reach in each direction as part of a training mode. Additionally, balance assessment and training device 1 may determine a score calculated based on the patient's limb length and average distances reached in each direction.

Once the assessment or training session concludes, the balance assessment and training device 1 can be powered down. The detachable rods 20 are then removed to render balance assessment and training device 1 more compact for convenient transportation and storage.

In another embodiment, previously-described rod 20 is replaced by a pair of parallel rods 38 retained within proximal rod end cap 28 and distal rod end cap 36 as depicted in FIG. 6. Linear bearings 40 retained within moveable slider 24 are provided to allow moveable slider 24 to move freely when pushed by the user's foot, but desirably restrict rotational movement of the moveable slider 24, thereby further preventing moveable slider 24 from contacting the ground during evaluation. In an additional embodiment, rod 20 may provide a hemispherical bearing groove (unillustrated) within the length of rod 20 with a complimentary hemispherical recess (also unillustrated) capturing a ball bearing, thereby also restricting moveable slider 24 from rotating about rod 20.

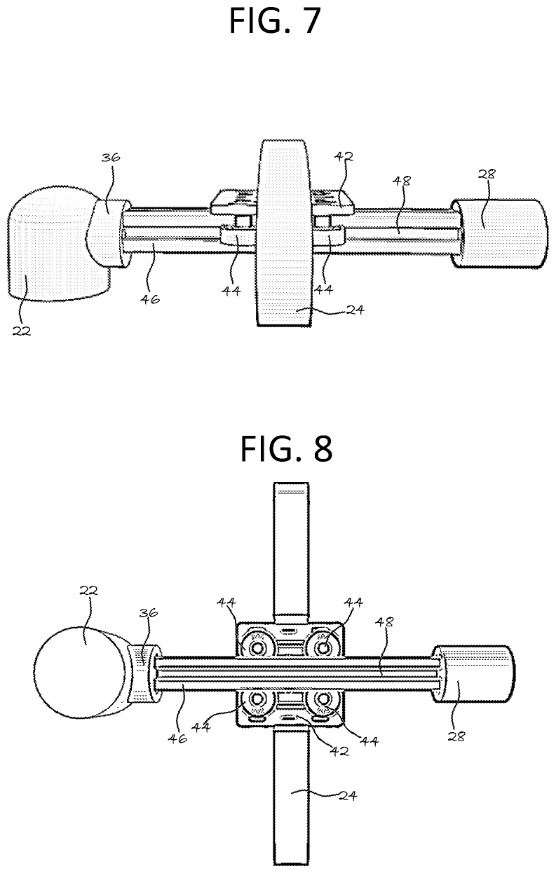

In a further embodiment shown in FIGS. 7 and 8, rod 20 is configured to provide a square cross-section depicted by rod 46 featuring two bearing grooves 48 on opposing sides. Moveable slider 24 provides gantry plate 42 with ball bearings 44 that travel within bearing grooves 48, again allowing moveable slider 24 to move freely when pushed by the user's foot, but desirably restricting rotational movement of the moveable slider 24 to prevent moveable slider 24 from contacting the ground during evaluation.

While the present invention has been described with respect to a single preferred embodiment, those skilled in the art will appreciate that a number of modifications and variations therefrom will be possible and it is intended to cover within the appended claims all such modifications and variations as come within the true spirit and scope of the present invention.

What is claimed is:

1. A device for assessing dynamic balance of a human patient, comprising:
a central platform; and
three rods;
said central platform having a top surface configured for the human patient to step on with one foot and comprising three engagement portions, each engagement portion being configured to detachably install a proximal end of one of said rods so as to assemble said dynamic balance assessing device, said engagement portions being oriented so as to arrange said rods at substantially even intervals in a plane around said central platform;
each of said rods comprising a moveable slider configured to be displaced a distance along a length of the rod by force applied by the patient's other foot;
said dynamic balance assessing device comprising a microcontroller, and a laser distance sensor configured to determine the distance a moveable slider was displaced along the length of the rod, wherein
a distal end of said rod comprises an axially oriented support configured to elevate said moveable sliders above the ground when said rods are installed within said engagement portion and said dynamic balance assessing device is placed on the ground.

2. The dynamic balance assessment device according to claim 1, wherein said central platform comprises a pressure sensor adapted to monitor pressure applied by the patient's foot, and
said microcontroller is configured to determine variation in pressure applied by said patient's foot while dynamic balance is being assessed.

3. The dynamic balance assessment device according to claim 1, wherein said central platform comprises a display screen configured to display the distances the patient displaces the moveable sliders.

4. The dynamic balance assessment device according to claim 2, wherein said central platform comprises a display screen configured to display the distances the patient displaces the moveable sliders, and
said dynamic balance assessing device is further configured to report when the variation in pressure applied by said patient's foot exceeds a predetermined level.

5. The dynamic balance assessment device according to claim 1, wherein said engagement portions and the proximal ends of said rods both comprise a permanent magnet, and said rods are configured to be detachably retained within said central platform by magnetic force.

6. The dynamic balance assessment device according to claim 1, wherein the proximal ends of said rods are detachably retained within said central platform by interference fit with said engagement portions.

7. The dynamic balance assessment device according to claim 1, wherein the proximal ends of said rods and said engagement portions comprise complimentary grooved threads, and said rods are detachably retained within said central platform by threading said proximal ends into said engagement portions.

8. The dynamic balance assessment device according to claim 1, wherein said proximal ends of said rods comprise an outwardly axially biased button,
each of said engagement portions comprises an aperture configured to receive said button, and
said engagement ends of said rods are detachably retained within said central platform by said axially biased button protruding through a corresponding aperture.

9. The dynamic balance assessment device according to claim 1, wherein said microcontroller is configured to wirelessly communicate the distances the patient displaces the sliders.

10. The dynamic balance assessment device according to claim 2, wherein said microcontroller is configured to wirelessly communicate the distances the patient displaces the sliders, and
said dynamic balance assessing device is further configured to wirelessly communicate when the variation in pressure applied by said patient's foot exceeds a predetermined level.

11. The dynamic balance assessment device according to claim 1, wherein each of said rods comprises a pair of parallel rods.

12. The dynamic balance assessment device according to claim 1, wherein each of said rods comprises a hemispherical bearing groove along the length of the rod,
said moveable slider comprises a complimentary hemispherical recess capturing a ball bearing, whereby
the moveable slider is restricted from rotating about the rod.

13. The dynamic balance assessment device according to claim 1, wherein each of said rods has a square cross-section with bearing grooves on a pair of opposing sides thereof, and
said moveable slider comprises a gantry plate having bearings that travel within said bearing grooves.
